(19) United States
(12) Patent Application Publication
(10) Pub. No.: US 2007/0135651 A1
(43) Pub. Date: Jun. 14, 2007

(54) Polymer-stabilised, crystallised, catanionic membranes, preparation method thereof and applications of same

(76) Inventors: Monique Dubois, Orsay (FR); Thomas Zemb, Viroflay (FR)

Correspondence Address: KNOBBE MARTENS OLSON & BEAR LLP, 2040 MAIN STREET, FOURTEENTH FLOOR, IRVINE, CA, 92614, US

(21) Appl. No.: 10/588,529

(22) PCT Filed: Feb. 14, 2005
(86) PCT No.: PCT/FR05/00342

§ 371 Date: Oct. 16, 2006

(30) Foreign Application Priority Data
Feb. 17, 2004 (FR) 0401579

(51) Int. Cl. C07C 233/00

(52) U.S. Cl. 554/56

(57) ABSTRACT

The invention relates to crystallised, catanionic membranes in organised solid bilayer form, having laterally-alternating anionic surfactants (AS) with H⁺ counter ions and cationic surfactants (CS) with OH⁻ counter ions which are co-crystallised with a mole fraction Q_{AS}/(Q_{AS}+Q_{CS}) greater than 0.5. According to the invention, the membrane forms a surface which is flat, at least locally, and the bilayer is stabilised by at least one polymer which is neutral and hydrophobic or which has an opposite overall electric charge to the effective charge of the catanionic membrane, the polymer being absorbed on the surface. The invention also relates to a method of preparing the membranes, the uses thereof, for examples as a medicament for the vectorisation of active species or for the retention of volatile molecules.

The present invention relates to polymer-stabilized crystalline catanionic membranes, to the process for preparing them and to the uses thereof, especially as medicaments for vectorizing active species or for retaining, via adsorption, volatile molecules.

Mixtures of anionic and cationic surfactants in aqueous medium give rise to what may conveniently be referred to as “catanionic” solutions.

After ionic pairing, the counterions form a salt in excess and induce high conductivity of the samples that masks the electrostatic interactions. One particular type of salt-free catanionic formulation is obtained by using only H⁺ and OH⁻ counterions, such that no excess of salt is formed by the mixing of the two surfactants (Dubois M. et al., C. R. Acad. Sci. Paris II C, 1998, 1(9) 567-565). The resulting catanionic systems are commonly known as “true catanionic systems”.

When these catanionic solutions are heated to a temperature above the melting point of the chains, the anionic and cationic surfactants self-assemble in the form of stable micelles of varied forms (spheres, cylinders or folded bilayers). Depending on the relative proportions between cationic and anionic constituents, various forms of structures may then be obtained during the cooling of these solutions.

When the catanionic solution contains an excess of cationic surfactants, the formation of crystalline nanodisks is observed, which are formed from a sandwich structure with a rigid outer membrane, the adjustable diameter of which may range from a few microns to about thirty nanometers and in which the positive charges are mainly located in the slice. The structure and the process for preparing these catanionic nanodisks are especially described in the article by Zemb T. et al., Science, 1999, 283, 816-819.

Conversely, when the catanionic solution contains an excess of anionic surfactants, the formation of hollow polyhedra is observed, the shape of which will vary as a function of the amount of anionic surfactants in excess. Under certain conditions, the formation of hollow icosahedra is observed in particular, the shape of which is reminiscent of that observed for viral capsid proteins. Their structure and the process for preparing them are especially described in the article by Dubois M. et al., Nature, 2001, 411, 672-675. Said article especially describes micrometer-sized icosahedra weighing about 10¹⁰ daltons, the structure of which is partly stabilized by the presence of pores at the apices of the polyhedra. According to said article, the formation of an icosahedron requires the conjunction of the following three conditions:

1) the formation of stable unilamellar vesicles during the high-temperature equimolar mixing of the anionic and cationic surfactant solutions,

2) the excess of anionic surfactant must be insoluble in water and in the crystalline bilayer obtained during the equimolar mixing of the anionic and cationic surfactants,

3) the amount of surfactant in excess must be such that it allows the formation of 10 to 15 pores per vesicle.

The absence of condition 3) leads to the formation of open crystalline large bilayers or nanodisks including pores.

Still according to the teaching of said article and by virtue of the presence of these pores, various uses of such polyhedra may be envisioned. They may be used, for example, as medicaments for the controlled release of active principles or of DNA in gene therapy or the isolation of RNA strands in order to protect them against the action of destructive enzymes. However, these polyhedra have the major drawback of being particularly sensitive to the slightest presence of salts, and have a tendency to aggregate together, which prohibits, for example, their use in physiological media, for instance in blood, and consequently their intravenous injection.

Moreover, various structures for delivering active principles, including sustained-release forms, have already been proposed and are based on the encapsulation of active ingredients inside vesicles. Thus, with this aim, a large number of prior art documents have described the use of spherical vesicles consisting of one or more lipid bilayers commonly denoted as liposomes. However, the use of liposomes is not always entirely satisfactory in terms of stability especially, and also since their preparation involves processes that require the use of organic solvents, the use of which is not necessarily compatible with physiological media and a particular material.

Thus, in order to overcome all these drawbacks and to provide a novel system for delivering active molecules that is stable, resistant to high ionic strengths and simple to use, the Inventors have developed that which forms the subject of the present invention.

A first subject of the invention is thus a catanionic membrane in the form of an organized solid bilayer comprising a lateral alternation of anionic surfactants with H⁺ counterions and of cationic surfactants with cocrystallized OH⁻ counterions in which the mole fraction (MF): molar amount of anionic surfactants (Q_{AS})/(molar amount of anionic surfactants (Q_{AS})+molar amount of cationic surfactants (Q_{CS})) is greater than 0.5 (i.e. Q_{AS}/(Q_{AS}+Q_{CS})>0.5), said membrane forming a surface that is at least locally flat, characterized in that said bilayer is stabilized with at least one polymer that is neutral and hydro-phobic or of overall electrical charge opposite the effective charge of said catanionic membrane, said polymer being adsorbed onto said surface.

The presence of the polymers absorbed onto their surface allows the catanionic membranes in accordance with the invention to be stabilized during Brownian motion, and in particular allows a distance of at least one nanometer to be conserved between two membranes, avoiding their precipitation and thus allowing their dilution with isotonic solutions such as seawater or blood.

The cationic and anionic surfactants that may be used in accordance with the invention are preferably chosen from compounds with a melting point greater than the working temperature so as to be in crystalline form. Since the working temperature may range between 20 and 30° C., surfactants with a melting point of greater than 30° C. will more particularly be selected.

The cationic surfactants that may be used in accordance with the invention for the formation of the bilayers are preferably chosen from the monocatenary and bicatenary quaternary ammoniums of formulae (I) and (I′), respectively, below:

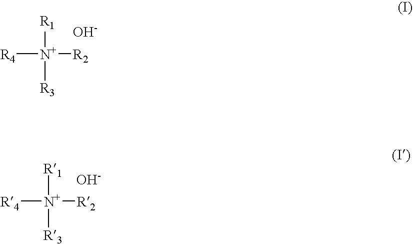

in which:
R₁, R₂ and R₃, which may be identical or different, represent a C₁-C₄ alkyl, C₁-C₄ hydroxyalkyl or (C₁-C₄)alkyl ether radical,
R′₁ and R′₂, which may be identical or different, represent a C₁-C₄ alkyl, C₁-C₄ hydroxyalkyl or (C₁-C₄)alkyl ether radical,
R′₃ and R′₄, which may be identical or different, represent a saturated or unsaturated C₈-C₂₄ hydrocarbon-based chain, a benzyl or (C₄-C₂₀)alkylbenzyl radical or a (C₄-C₂₀)alkyl ester group,
R₄ represents a saturated or unsaturated C₈-C₂₄ hydrocarbon-based chain, a benzyl or (C₄-C₂₀)alkylbenzyl radical or a (C₄-C₂₀)alkyl ester group;
   and mixtures thereof.

Among the C₁-C₄ alkyl radicals of the cationic surfactants of formulae (I) and (I′) above, the methyl radical is particularly preferred.

Among the C₈-C₂₄ hydrocarbon-based chains of the radicals R′₃, R′₄ and R′₄, mention may be made especially of alkyl chains, for instance stearyl, cetyl, dodecyl and tetradecyl chains.

Among the (C₄-C₂₀)alkyl ester groups mentioned for the radicals R′₃, R′₄ and R₄, mention may be made in particular of (C₁₆)alkyl esters and (C₁₂)alkyl esters.

Among the compounds of formula (I) above with OH⁻ counterions, mention may be made in particular of cetyltrimethylammonium hydroxide, dodecyltrimethyl-ammonium hydroxide, stearyltrimethylammonium hydroxide, tetradecyltrimethylammonium hydroxide, N-(2-carboxyethyl)-N,N-dimethyl-1-hexadecanaminium hydroxide, N-(2-hydroxyethyl)-N,N-dimethyl-1-hexadecanaminium hydroxide, cetyltriethylammonium hydroxide, dodecyltriethylammonium hydroxide, stearyl-triethylammonium hydroxide, tetradecyltriethylammonium hydroxide, cetyltripropylammonium hydroxide, dodecyl-tripropylammonium hydroxide, stearyltripropylammonium hydroxide and tetradecyltripropylammonium hydroxide.

Among the compounds of formula (I′) above, mention may be made in particular of didodecyldimethylammonium hydroxide, didodecyldiethylammonium hydroxide, didodecyldipropylammonium hydroxide, didodecyldibutylammonium hydroxide and dicetyldimethyltrimethylammonium hydroxide.

The anionic surfactants that may be used in accordance with the invention for the formation of the bilayers are preferably chosen from carboxylic acids with a C₈-C₂₄ carbon-based hydrophobic chain with H⁺ counterions and phosphates and sulfonates with H⁺ counterions comprising one or two C₁₂-C₂₀ alkyl chains.

Among these anionic surfactants, mention may be made in particular of fatty acids such as myristic acid, lauric acid and palmitic acid, phosphates, sulfonates, benzyl sulfates and monocatenary glycerol monoesters, preferably having a fine X-ray diffraction peak at large angles after combination with the cationic surfactant, located at q=1.52±0.15 Å⁻¹ without a shoulder, as described, for example, by Rank J. L. et al., J. Mol. Biol., 1974, 85(2), 249-277.

It is possible, in accordance with the invention, to combine any type of anionic surfactant with H⁺ counterions with any type of cationic surfactant with OH⁻ counterions and in particular with any cationic surfactant of formula (I) and/or of formula (I′) since it is possible to envision mixing of monocatenary and bicatenary cationic surfactants.

As has been indicated previously, one of the essential characteristics of the invention is that the MF of the surfactants used for the formation of the bilayers (Q_{AS}/(Q_{AS}+Q_{CS})) should be greater than 0.5. According to one preferred embodiment of the invention, this MF is between 0.52 and 0.66 and even more preferentially between 0.55 and 0.58.

According to one particular and preferred embodiment of the invention, the bilayers consist:

a) either of a cationic surfactant of formula (I) as defined above and in which the radicals R₁, R₂ and R₃ are identical and represent a methyl radical and R₄ represents a hydrocarbon-based chain containing X carbon atoms, X being between 8 and 24 inclusive, combined with a carboxylic acid as defined above in which the C₈-C₂₄ carbon-based hydrophobic chain contains X±4 carbon atoms;

b) or of a cationic surfactant of formula (I′) as defined above in which the radicals R′₁ and R′₂ are identical and represent a methyl radical and R′₃ and R′₄ are identical and represent a hydrocarbon-based chain containing X carbon atoms, X being between 8 and 24 inclusive, combined with a carboxylic acid as defined above in which the C₈-C₂₄ carbon-based hydrophobic chain contains X±4 carbon atoms;

c) or a phosphate or a sulfonate comprising two identical alkyl chains containing X carbon atoms, X being between 8 and 24 inclusive, combined with a cationic surfactant of formula (I) as defined above and in which the radicals R₁, R₂ and R₃ are identical and represent a methyl radical and R₄ represents a C₈-C₂₄ alkyl chain;

d) or a phosphate or a sulfonate comprising only one alkyl chain containing X carbon atoms, X being between 8 and 24 inclusive, combined with a cationic surfactant of formula (I′) as defined above and in which the radicals R′₁ and R′₂ are identical and represent a methyl radical and R′₃ and R′₄ are identical and represent a C₈-C₂₄ alkyl chain.

According to an even more particularly preferred form of the invention, the bilayers are formed from a combination of cetyltrimethylammonium with OH⁻ counterions and of myristic acid with H⁺ counterions.

In addition to the anionic surfactants with H⁺ counterions, the bilayers in accordance with the invention may also contain a minor molar amount of anionic surfactants with metal counterions, and in particular with a sodium, magnesium, lithium, chromium, vanadium or nickel counterion, for example, said surfactants being chosen from the anionic surfactants mentioned above, with the exception, of course, of the nature of the counterion. According to the invention, the term “minor” molar amount means an amount of anionic surfactant with a metallic counterion that is necessary to bring the final MF within the range of between 0.52 and 0.66 as defined above.

According to one particular embodiment of the invention, the pairs of ions forming the polar heads of the cationic and anionic surfactants perpendicularly occupy an area preferably equal to that of the crystalline chains, on a hexagonal network, which may be detected by a first fine peak located at about q=1.52±0.15 Å⁻¹.

According to one advantageous embodiment of the invention, the polymers that may be used to stabilize the bilayers in accordance with the invention are nonlipid polymers, chosen from polymers that are neutral or that have an overall electrical charge opposite the effective charge of the catanionic membranes, i.e. of “weakly” negative overall electrical charge.

For the purposes of the present invention, the term “effective charge” means the overall electrical charge taking into account the apparent pKa of the acid in the crystal, which is manifested, for example, in electrophoretic mobility by a movement of the bilayers towards the anode. This electrical charge is different than the assayable structural charge and deduced from the localization of the composition in a phase diagram.

Also for the purposes of the present invention, the term “polymers of “weakly” negative overall electrical charge means polymers comprising less than one elemental electrical charge per 2 nano-meters of drawn length.

When they are neutral polymers, they are preferably chosen from polysaccharides, for instance dextrans and cellulose derivatives such as hydroxymethylcelluloses, hydroxyethylcelluloses and hydroxypropylcelluloses, synthetic polymers such as polyethylene glycols (PEG), polyoxyethylenes, polyvinylpyrrolidone (PVP) and polyvinyl alcohols such as the products sold under the trade names PVA, Ethenol®, Poval®, Acroflex®, Airvol®, Alcotex® or Aquafilm®, oxyethylenated diblock polymers such as the polymers sold under the trade name Varonic® by the company Degussa-Goldschmidt, block copolymers based on ethylene oxide and propylene oxide, such as the polymers sold under the trade names Pluronic® and Lutrol® by the company BASF, and water-soluble equivalents thereof, water-soluble triblock copolymers, i.e. copolymers composed of hydrophilic-hydrophobic-hydrophilic blocks, such as the products sold under the trade names Methyl oxirane, EOPO copolymer, Antarox®, Arcol®, Daltocel® or Dowfax® and analogs thereof comprising polystyrene as hydrophobic group.

When they are polymers of weakly negative overall electrical charge, they are preferably chosen from polyacrylates, polymethacrylates, polyethyl methacrylates, polybutyl methacrylates and polystyrene-sulfonates, said polymers being substituted to more than 75% randomly with neutral water-soluble groups, for instance polyoxyethylene groups or the like.

Among the polymers that may be used in accordance with the invention, it is most particularly preferred to use weakly adsorbed polymers such as polyoxyethylene, dextran, PVP or the polymers sold under the trade names Varonic®, Pluronic® and Lutrol®, Methyl Oxirane, Pluronic®, Antarox®, Arcol®, Daltocel® and Dowfax®.

Among these polymers, it is even more particularly preferred to use a polyethylene glycol with a molecular mass of between 5000 and 50 000 Da and even more preferentially between 10 000 and 20 000 Da.

These polymers preferably represent from 10% to 400% and even more particularly from 100% to 200% by weight relative to the total weight of the bilayer.

According to a particular embodiment of the invention, the catanionic membranes may especially be in the form of faceted hollow microcrystals when the MF Q_{AS}/(Q_{AS}+Q_{CS}) is between 0.55 and 0.58.

In this case, these microcrystals may take the form of hollow polyhedra (molecular boxes) comprising from 12 to 30 approximately triangular faces, and most particularly in the form of hollow icosahedra with an inner volume of between 0.1 and 10 μ³.

Within the organized solid bilayer of each of the faces of these microcrystals, the lateral alternation of the cocrystallized anionic and cationic surfactants is hexagonal, the flat part of said faces consisting solely of species containing H⁺ or OH⁻ counterions in stoichiometric amounts, whereas the apices of said faces are in the form of an internal semitorus predominantly formed from the anionic species in excess and in an amount sufficient to obtain an MF Q_{AS}/(Q_{AS}+Q_{CS}) of between 0.55 and 0.58.

According to this configuration, i.e. when the apices of each face are in the form of an internal semitorus, then the apex of each of the faces of a microcrystal forms a pore, together with the apices of the adjacent faces of the same microcrystal. In this case, each microcrystal may comprise from about 10 to 15 pores.

By virtue of the presence of the neutral polymers or polymers with a weakly negative electrical charge adsorbed onto their surface, which block flocculation and coalescence, these molecular boxes can withstand the ionic strength, i.e. the presence of salts, up to the point of isotonicity. The presentation of the catanionic membranes in molecular-box form is particularly preferred according to the invention.

The catanionic membranes in accordance with the invention may also be in the form of fragments of hollow polyhedra, i.e. in the form of a stack of three-dimensional catanionic crystals in the form of a “pile of plates”, resulting from the opening of the molecular boxes and the dense stacking of the fragments of the facets.

After their formation and before the adsorption of the specific stabilizing polymer, the pH of the membrane solution may be adjusted to any pH value of between 2 and 6. This allows reactions to be performed in acidic medium and also prevents the precipitation of macroscopic crystals. To do this, acids with hydrophilic counterions will preferably be used, for instance hydrochloric acid, acetic acid and citric acid.

These catanionic membranes are stable in physiological media and make it possible especially to retain by adsorption and/or encapsulation and to control the slow diffusion of pharmaceutical or cosmetic active molecules, or alternatively of cells such as bacteria, for example. They are capable in particular of withstanding high osmotic compressions and even of withstanding the conversion of water into ice, while at the same time remaining in the form of clearly individualized aggregates with rigid and locally flat walls.

A subject of the present invention is also a process for preparing a catanionic membrane in accordance with the invention and as described above, said process being characterized in that it comprises the following steps:

1) a first step of formation of unilamellar vesicles by mixing, in an aqueous solvent of low conductivity:
a) a cationic surfactant (CS) with OH⁻ counterions in a molar amount Q_{CS} and
b) one or more anionic surfactants (AS) in a molar amount Q_{AS} strictly greater than Q_{CS}, and corresponding to equations (1) to (3) below:
   Q_{AS}=Q_{AS1}+Q_{AS2} (1)
   Q_{AS1}=Q_{CS} (2) and
   Q_{AS2}<2(Q_{CS}) (3)
in which:
   Q_{AS1} is the molar amount of an anionic surfactant with H⁺ counterions
   Q_{AS2} is the molar amount of an anionic surfactant with H⁺ counterions or with metal counterions, said surfactants having a carbon-based chain identical to that of the CS or of the AS with H⁺ counterions used in an amount Q_{AS1},

said mixture of cationic surfactant and of anionic surfactant being prepared at a temperature above the melting point of the chains of said surfactants;

2) a second step of obtaining flat aggregates formed from only one interdigitated or non-inter-digitated crystalline molecular bilayer, by cooling the mixture obtained in the first step to a temperature below the melting point of the chains of the surfactants present in the mixture;

3) a third step of stabilizing the crystalline molecular bilayers obtained above in the second step, by adding at least one neutral and hydrophobic polymer or a polymer of weakly negative overall electrical charge dissolved in an aqueous solvent of low conductivity, said step being performed at a temperature below the melting point of the chains of the surfactants present in the mixture.

During the first step, the dissolution of the surfactants in the aqueous solvent is preferably performed slowly and may especially take place over variable times ranging from one hour to one week, with minimum mechanical stirring, without heating.

According to a first particular embodiment of this process, and when the anionic surfactants used during the first step consist only of AS with H⁺ counterions, this is then referred to as a true catanionic mixture thus comprising only OH⁻ counterions (provided by the CSs) and H⁺ counterions. In this case, the mixture of the ASs and the CSs may optionally be prepared beforehand in powder form before dissolving in the solvent.

According to a second embodiment of this process, and when the excess AS consists of ASs with metal counterions, then the first step of the process in accordance with the invention comprises:
a first substep in which the CS with OH⁻ counterions is first mixed with the AS with H⁺ counter-ions in an amount Q_{AS1} equal to Q_{CS}, and then
a second substep in which the molar amount Q_{AS2} of AS with metal counterions is then added.

In this case, the nature of the metal counterions may be chosen as a function of the property that it is desired to give to the cationic membrane in accordance with the present invention. By way of example, mention may be made especially of sodium counterions with anticorrosive properties, and also chromium, vanadium and nickel ions, which have electro-chemical corrosion-inhibiting properties on iron alloys.

Once the surfactant solutions are optically homogeneous, the heating step makes it possible to completely melt the chains of the surfactants associated with the pairs of ions formed during the slow dissolution. During this step, the compounds intended to form the bilayers in accordance with the invention are dispersed in the form of vesicles with high electrostatic repulsion. Each vesicle constitutes a microreactor formed from a fluid bilayer (liquid chains), which, after the cooling step, will be converted into a rigid bilayer (gelled chains).

The aqueous solvents used during this process preferably have a conductivity of less than or equal to about 1 MOhm. They are preferably chosen from water and glycerol, and mixtures thereof.

During the first step, the total concentration of surfactants (AS+CS) in the solution is preferably between 0.01% and 3% by weight relative to the total weight of said solution.

Also during the first step, the temperature to which the mixture is heated obviously depends on the nature of the cationic and anionic surfactants used. However, in general, this temperature is generally greater than 30° C. and less than 80° C. and even more preferentially between 30° C. and 70° C. This temperature may be adjusted for each surfactant solution, to 5° C. above the temperature of the mixture of surfactants under consideration, which may be detected by an endothermic peak by differential scanning calorimetry (DSC).

This temperature is even more preferentially between 55° C. and 70° C. Thus, during the second step, the mixture is cooled to a temperature preferably less than 30° C. and even more preferentially to a temperature of between 20° C. and 25° C.

According to one particular embodiment of the process in accordance with the invention, at least one active substance that is absorbed onto the surface of the catanionic membranes and/or encapsulated inside the vesicles (molecular boxes) may also be added to the mixture during the first step. Among these active substances, mention may be made especially of pharmaceutical active principles, active substances for cosmetic purposes and in particular volatile odoriferous molecules, cells such as whole bacteria, and DNA or RNA fragments.

At this moment, a person skilled in the art will preferably take care to select active substances whose electrical charge will be sufficiently low, so as to avoid any destabilization of the catanionic membranes in accordance with the invention.

According to one particular embodiment of the process in accordance with the invention, it is also possible, during the second step, to adjust the pH of the mixture to between 2 and 6 as indicated previously.

According to one advantageous embodiment of the invention and when the catanionic membranes are in the form of hollow polyhedra (molecular boxes), then the process generally includes an additional step of removing the active substances that have not been encapsulated in the polyhedra or that have been adsorbed onto their surface. This removal step may be performed by rinsing, especially using an aqueous solvent identical to the solvent used for the preparation of the membranes, by dialysis or alternatively by filtration.

According to the invention, the volume fraction of polymer added to the mixture during the third step is preferably between one and two times the total mass of the cationic and anionic surfactants in order to have a steric or electrostatic protective layer allowing the addition of salt without destroying the faceted polyhedra of icosahedron type, or the precipitation of catanionic species forming a three-dimensional crystal.

Finally, a subject of the invention is also the catanionic membranes in accordance with the invention and as described above, for use as medicament for the vectorization of active species or for the retention by adsorption and slow diffusion of volatile molecules.

According to one advantageous embodiment of the invention, the catanionic membrane is in the form of a faceted hollow polyhedron and is used for:
encapsulating medicaments, for the purpose of vectorizing them,
encapsulating whole bacteria or DNA or RNA fragments, so as to make them inaccessible to the immune system,
retaining reagents for chemical reactions taking place inside the polyhedra,
performing precipitation or crystallization reactions inside the polyhedra, by slow diffusion of reagents inside across the pores of the polyhedra,
as cosmetic ingredient for the manufacture of creams, obtained by flocculation in the form of bunches of polyhedra, and allowing the efficient diffusion of active molecules after adsorption of the polyhedron onto surfaces of opposite surface electrical potential, for instance the skin.

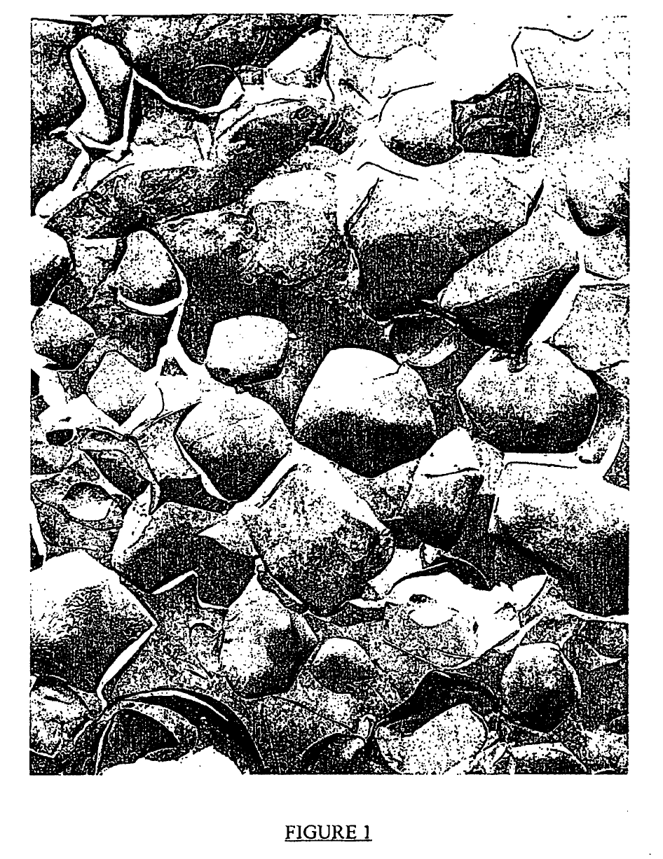

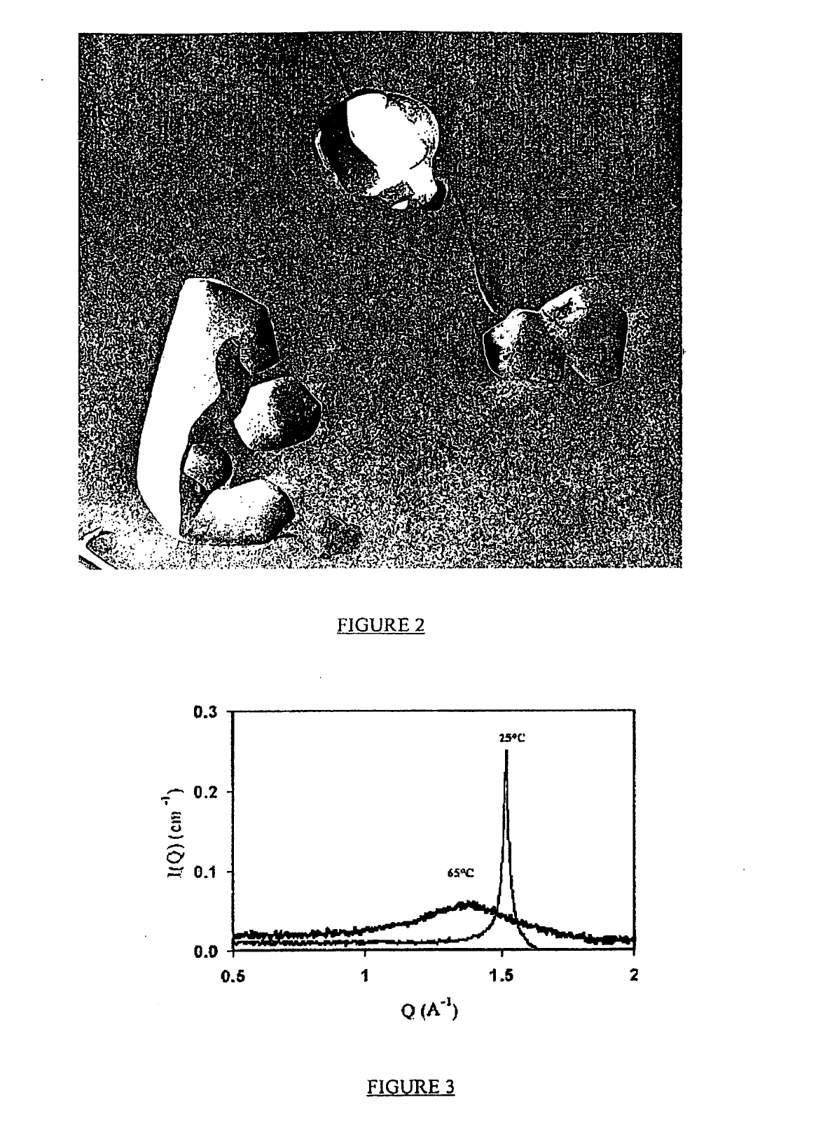

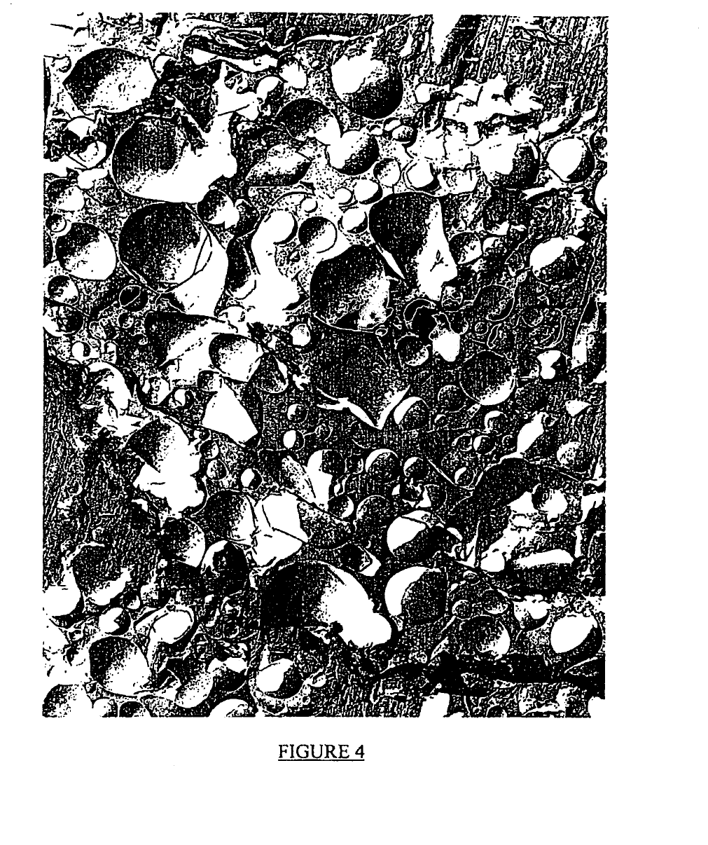

Besides the preceding arrangements, the invention also comprises other arrangements that will emerge from the description that follows, which refers to an example of preparation of crystalline hollow polyhedra based on cetyltrimethylammonium hydroxide and myristic acid with H⁺ counterions and an example of preparation of crystalline hollow polyhedra based on cetyltrimethylammonium hydroxide and myristic acid with H⁺ and Li⁺ counterions, and also to the attached FIGS. 1 to 4, in which:
FIG. 1 is a cryofracture microscopy photograph of crystalline hollow polyhedra based on cetyltrimethylammonium hydroxide and myristic acid with H⁺ counterions before stabilization with a neutral polymer (PEG 20 000) and at a total concentration of surfactants in water of 1% by weight and with a mole fraction AS/(AS+CS) equal to 0.56;
FIG. 2 is a cryofracture microscopy photograph of crystalline hollow polyhedra based on cetyltrimethylammonium hydroxide and myristic acid with H⁺ counterions after stabilization with a neutral polyethylene glycol polymer (PEG 20 000) and at a total concentration of surfactants in water of 1% by weight and with a mole fraction AS/(AS+CS) equal to 0.56;
the attached FIG. 3 shows the X-ray diffraction spectra at 25° C. (crystalline structure) and at 65° C. (noncrystalline structure) of catanionic mixtures of CTAOH and of myristic acid with H⁺ counter-ions, on which the diffusion intensity (in cm⁻¹) is expressed as a function of the wave vector Q (in Å⁻¹);
FIG. 4 is a cryofracture microscopy photograph of crystalline hollow polyhedra based on cetyltrimethylammonium hydroxide and myristic acid with H⁺ counterions in equimolar amounts, and of an excess of myristic acid with Li⁺ counterions.

It should be clearly understood, however, that these examples are given solely as illustrations of the subject of the invention, of which they do not in any way constitute a limitation.

### EXAMPLE 1

Preparation of Crystalline Hollow Polyhedra Based on Cetyltrimethylammonium Hydroxide and Myristic Acid with H⁺ Counterions

This example illustrates one of the two variants of the process for preparing the bilayers in accordance with the invention, namely that in which the CS with OH⁻ counterions and the AS with H⁺ counterions in excess are mixed together directly to obtain a true catanionic mixture, without passing via a prior substep of equimolar premixing of AS and CS.

To do this, 0.023 g of cetyltrimethylammonium hydroxide (CTAOH) and 0.022 g of myristic acid with H⁺ counterions are mixed together, in the form of freeze-dried powders. A mixture whose mole fraction of myristic acid with H⁺ counterions/(myristic acid with H⁺ counterions+CTAOH) is equal to 0.56 is thus obtained. 4.46 g of Millipore water (<1 MOhm/cm) is then added to the powder mixture thus obtained to form a solution with a total weight concentration of surfactants equal to 1% and a mole fraction AS/(AS+CS) equal to 0.56. The compounds are left to dissolve at room temperature with gentle stirring for one week. After the surfactants have fully dissolved (disappearance of the solid grains of myristic acid), the solution is heated to a uniform temperature and slightly higher than 65° C., which corresponds to the melting point of the chains of the surfactants, for one minute. The solution is then allowed to cool to room temperature.

Separately, a solution containing 1.5% by weight of polyethylene glycol sold under the name PEG 20 000 (neutral polymer) is prepared. The surfactant solution is mixed at room temperature, on a volume-for-volume basis, with the polymer solution with gentle stirring. The final dispersion obtained may be concentrated by simple filtration on account of the conjunction of the crystallization of the chains, the size of the objects, the low viscosity of the solution and the low osmotic pressure (<1000 Pa) of the final dispersion obtained.

The solution obtained before concentration is slightly scattering and bluish and contains a dispersion of hollow micron-sized faceted objects that may be detected by their characteristic scattering decreasing with the square of the scattering angle at small angles (light or neutrons), associated with the presence of a fine Bragg peak (<0.002 nm) located between 0.150 nm⁻¹ and 0.156 nm ⁻¹ by large-angle X-ray scattering.

Moreover, the stability properties of the membrane according to the invention in the presence of saline solutions up to isotonic osmolarity were demonstrated by mixing with a concentrated sodium chloride solution until a final saline concentration of 0.15 M was obtained.

The icosahedric boxes thus obtained were observed directly by cryofracture microscopy. The images obtained are given in the attached FIGS. 1 and 2, taken before and after stabilization with the neutral polymer. The crystalline nature of the faces was moreover confirmed by X-ray scattering on samples of identical chemical nature but more concentrated (band at q=1.52±0.01 Å⁻¹ as is seen in the attached FIG. 3 that shows the scattering intensity (in cm⁻¹) as a function of the wave vector Q (in Å⁻¹) at a temperature of 65° C. (noncrystalline structure) and at a temperature of 25° C. (crystalline structure).

### EXAMPLE 2

Preparation of Crystalline Hollow Polyhedra Based on Cetyltrimethylammonium Hydroxide and Myristic Acid with H⁺ Counterions and an Excess of Myristic Acid with Li⁺ Counterions

This example illustrates the second variant of the process in accordance with the invention, i.e. the preparation of hollow polyhedra in which the excess of anionic surfactant is composed of an anionic surfactant with lithium counterions, which imposes the implementation of the first step of the process in two substeps as explicitly described previously.

Just as in example 1, 0.0230 g of freeze-dried CTAOH and 0.0174 g of myristic acid with H⁺ counterions are first mixed together and an amount of Millipore water (>1 MOhm/cm) sufficient to form a solution containing 1% by weight of total surfactants is then added. In this solution, the molar amount Q_{C} of CTAOH is identical to the molar amount Q_{A1} of myristic acid with H⁺ counterions. The surfactant mixture is then allowed to dissolve by slow stirring at room temperature until a solution that no longer contains any heterogeneities visible to the naked eye and corresponding to myristic acid crystals is obtained. The solution is then heated to a temperature above 50° C. for 1 minute; the solution should be transparent and should not contain any aggregates. It is allowed to cool to room temperature. 0.0051 g of powdered lithium myristate is then added to this solution. A solution of catanionic surfactants is then obtained, in which the mole fraction of myristate (H⁺+Li⁺)/(myristate (H⁺+Li⁺)+CTAOH) is equal to 0.56. The mixture is stirred at room temperature for one week until the lithium myristate has fully dissolved. The catanionic solution is then heated to a temperature above 65° C. for 1 minute, and the solution is then allowed to cool to room temperature. The objects obtained by this method have the same structure as those of example 1 above and are shown in the attached FIG. 4 obtained by cryo-fracture microscopy.

Separately, a solution containing 1.5% by weight of neutral polymer: polyethylene glycol (PEG 20 000) is prepared. The catanionic solution is mixed at room temperature, volume-for-volume, with the neutral polymer solution with gentle stirring. As in example 1 above, the final dispersion obtained may be concentrated by simple filtration.

The structural signature (not shown) is similar to that of example 1, both by cryofracture and by X-ray.

1. A catanionic membrane in the form of an organized solid bilayer comprising a lateral alternation of anionic surfactants with H⁺ counterions and of cationic surfactants with cocrystallized OH⁻ counterions in which the mole fraction (MF): molar amount of anionic surfactants (Q_{AS})/(molar amount of anionic surfactants (Q_{AS})+molar amount of cationic surfactants (Q_{CS})) is greater than 0.5, said membrane forming a surface that is at least locally flat, wherein said bilayer is stabilized with at least one polymer that is neutral and hydrophobic or of overall electrical charge opposite the effective charge of said catanionic membrane, said polymer being adsorbed onto said surface.

2. The membrane as claimed in claim 1, wherein the cationic and anionic surfactants are chosen from surfactants with a melting point of greater than 30° C.

3. The membrane as claimed in claim 1, wherein the cationic surfactants are selected from the monocatenary and bicatenary quaternary ammoniums of formulae (I) and (I′), respectively, below: 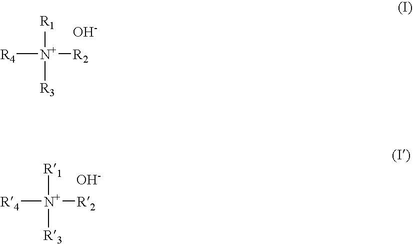
in which:
R₁, R₂ and R₃, which may be identical or different, represent a C₁-C₄ alkyl, C₁-C₄ hydroxyalkyl or (C₁-C₄)alkyl ether radical,
R′₁ and R′₂, which may be identical or different, represent a C₁-C₄ alkyl, C₁-C₄ hydroxyalkyl or (C₁-C₄)alkyl ether radical,
R′₃ and R′₄, which may be identical or different, represent a saturated or unsaturated C₈-C₂₄ hydrocarbon-based chain, a benzyl or (C₄-C₂₀)alkylbenzyl radical or a (C₄-C₂₀)alkyl ester group,
R₄ represents a saturated or unsaturated C₈-C₂₄ hydrocarbon-based chain, a benzyl or (C₄-C₂₀)alkylbenzyl radical or a (C₄-C₂₀)alkyl ester group;
and mixtures thereof.

4. The membrane as claimed in claim 3, wherein the C₁-C₄ alkyl radicals are methyl radicals.

5. The membrane as claimed in claim 3, wherein the compounds of formula (I) are selected from the group consisting of cetyltrimethylammonium hydroxide, dodecyltrimethylammonium hydroxide, stearyltrimethylammonium hydroxide, tetradecyltrimethylammonium hydroxide, N-(2-carboxyethyl)-N,N-dimethyl-1-hexadecanaminium hydroxide, N-(2-hydroxyethyl)-N,N-dimethyl-1-hexadecanaminium hydroxide, cetyltriethylammonium hydroxide, dodecyl-triethylammonium hydroxide, stearyltriethylammonium hydroxide, tetradecyltriethylammonium hydroxide, cetyltripropylammonium hydroxide, dodecyltripropylammonium hydroxide, stearyltripropylammonium hydroxide and tetradecyltripropylammonium hydroxide.

6. The membrane as claimed in claim 3, wherein the compounds of formula (I′) are selected from the group consisting of didodecyldimethylammonium hydroxide, didodecyldiethylammonium hydroxide, didodecyldipropylammonium hydroxide, didodecyldibutylammonium hydroxide and dicetyldimethyltrimethylammonium hydroxide.

7. The membrane as claimed in claim 1, wherein the anionic surfactants are selected from the group consisting of carboxylic acids with a C₈-C₂₄ carbon-based hydrophobic chain with H⁺ counterions and phosphates and sulfonates with H⁺ counterions comprising one or two C₁₂-C₂₀ alkyl chains.

8. The membrane as claimed in claim 7, wherein the anionic surfactants are selected from the group consisting of myristic acid, lauric acid and palmitic acid, phosphates, sulfates, benzyl sulfates and monocatenary glycerol monoesters.

9. The membrane as claimed in claim 3, wherein the bilayers consist of:
a) a cationic surfactant of formula (I) as defined in claim 3 and in which the radicals R₁, R₂ and R₃ are identical and represent a methyl radical and R₄ represents a hydrocarbon-based chain containing X carbon atoms, X being between 8 and 24 inclusive, combined with a carboxylic acid having a C₈-C₂₄ carbon-based hydrophobic chain with H⁺ counterions in which the C₈-C₂₄ carbon-based hydrophobic chain contains X±4 carbon atoms;
b) a cationic surfactant of formula (I′) as defined in claim 3 in which the radicals R′₁ and R′₂ are identical and represent a methyl radical and R′₃ and R′₄ are identical and represent a hydrocarbon-based chain containing X carbon atoms, X being between 8 and 24 inclusive, combined with a carboxylic acid having a C₈-C₂₄ carbon-based hydrophobic chain with H⁺ counterions in which the C₈-C₂₄ carbon-based hydrophobic chain contains X±4 carbon atoms;
c) a phosphate or a sulfonate comprising two identical alkyl chains containing X carbon atoms, X being between 8 and 24 inclusive, combined with a cationic surfactant of formula (I) as defined in claim 3 and in which the radicals R₁, R₂ and R₃ are identical and represent a methyl radical and R₄ represents a C₈-C₂₄ alkyl chain; or
d) a phosphate or a sulfonate comprising only one alkyl chain containing X carbon atoms, X being between 8 and 24 inclusive, combined with a cationic surfactant of formula (I′) as defined in claim 3 and in which the radicals R′₁ and R′₂ are identical and represent a methyl radical and R′₃ and R′₄ are identical and represent a C₈-C₂₄ alkyl chain.

10. The membrane as claimed in claim 9, wherein the bilayers are formed from a combination of cetyltrimethylammonium with OH⁻ counterions and myristic acid with H⁺ counterions.

11. The membrane as claimed in claim 1, wherein the mole fraction Q_{AS}/(Q_{AS}+Q_{CS}) is between 0.52 and 0.66.

12. The membrane as claimed in claim 1, wherein the bilayers further comprise a minor molar amount of anionic surfactants with metal counterions.

13. The membrane as claimed in claim 1, wherein the neutral polymers are nonlipid polymers selected from the group consisting of polysaccharides, polyethylene glycols, polyoxy-ethylenes, polyvinylpyrrolidone, polyvinyl alcohols, oxyethylenated diblock polymers, block copolymers based on ethylene oxide and propylene oxide, and triblock copolymers composed of hydrophilic-hydrophobic-hydrophilic blocks.

14. The membrane as claimed in claim 1, wherein the polymers with an overall electrical charge opposite the effective charge of the catanionic membranes are polymers of weakly negative electrical charge selected from the group consisting of polyacrylates, polymethacrylates, polyethyl methacrylates, polybutyl methacrylates and polystyrenesulfonates, said polymers being substituted to more than 75% randomly with neutral water-soluble groups.

15. The membrane as claimed in claim 14, wherein said polymer is a polyethylene glycol with a molecular mass of between 5000 and 50 000 Da.

16. The membrane as claimed in claim 1, wherein said polymers represent from 10% to 400% by weight relative to the total weight of the bilayer.

17. The membrane as claimed in claim 1, wherein the mole fraction Q_{AS}/(Q_{AS}+Q_{CS}) is between 0.55 and 0.58 and is in the form of faceted hollow microcrystals.

18. The membrane as claimed in claim 17, wherein the membrane is in the form of hollow polyhedra comprising from 12 to 30 approximately triangular faces.

19. The membrane as claimed in claim 18, wherein the membrane is in the form of hollow icosahedra with an inner volume of between 0.1 and 10 μ³.

20. The membrane as claimed in claim 18, wherein, within the organized solid bilayer of each of the faces of said microcrystals, the lateral alternation of the cocrystallized anionic and cationic surfactants is hexagonal, the flat part of said faces consisting solely of species containing H⁺ or OH⁻ counterions in stoichiometric amounts, whereas the apices of said faces are in the form of an internal semitorus predominantly formed from the anionic species in excess and in an amount sufficient to obtain a MF Q_{AS}/(Q_{AS}+Q_{CS}) of between 0.55 and 0.58.

21. The membrane as claimed in claim 20, wherein the apex of each of the faces of a microcrystal forms a pore, together with the apices of the adjacent faces of the same microcrystal.

22. The membrane as claimed in claim 17, wherein the membrane is in the form of fragments of hollow polyhedra constituting a stack of three-dimensional catanionic crystals in the form of a “pile of plates”.

23. A method for preparing a catanionic membrane as defined in claim 1, comprising the following steps:
1) forming unilamellar vesicles by mixing, in an aqueous solvent of low conductivity:
a) a cationic surfactant (CS) with OH⁻ counterions in a molar amount Q_{CS} and
b) one or more anionic surfactants (AS) in a molar amount Q_{AS} strictly greater than Q_{CS}, and corresponding to equations (1) to (3) below:
Q_{AS}=Q_{AS1}+Q_{AS2} (1)
Q_{AS1}=Q_{CS} (2) and
Q_{AS2}<2(Q_{CS}) (3)
in which:
Q_{AS1} is the molar amount of an anionic surfactant with H⁺ counterions
Q_{AS2} is the molar amount of an anionic surfactant with H⁺ counterions or with metal counterions, said surfactants having a carbon-based chain identical to that of the CS or of the AS with H⁺ counterions used in an amount Q_{AS1},
said mixture of cationic surfactant and of anionic surfactant being prepared at a temperature above the melting point of the chains of said surfactants;
2) cooling the mixture obtained in the first step to a temperature below the melting point of the chains of the surfactants present in the mixtures, thereby obtaining flat aggregates formed from only one interdigitated or noninterdigitated crystalline molecular bilayer; and
3) adding at least one neutral and hydrophobic polymer or a polymer of weakly negative overall electrical charge dissolved in an aqueous solvent of low conductivity, thereby stabilizing the crystalline molecular bilayers obtained above in the second step, said step being performed at a temperature below the melting point of the chains of the surfactants present in the mixture.

24. The method as claimed in claim 23, characterized in that, when the excess of anionic surfactant consists of anionic surfactants with metal counterions, then the first step of the method comprises:
a first substep in which the cationic surfactant with OH⁻ counterions is first mixed with the anionic surfactant with H⁺ counterions in an amount Q_{AS1} equal to Q_{CS}, and then
a second substep in which the molar amount Q_{AS2} of anionic surfactant with metal counterions is then added.

25. The method as claimed in claim 23, wherein the aqueous solvents have a conductivity of less than or equal to 1 MOhm.

26. The method as claimed in claim 23, wherein the aqueous solvents are selected from the group consisting of water and glycerol, and mixtures thereof.

27. The method as claimed in claim 23, wherein, during the first step, the total concentration of surfactants in the solution is between 0.01% and 3% by weight relative to the total weight of said solution.

28. The method as claimed in claim 23, wherein, during the first step, the temperature is greater than 30° C. and less than 80° C.

29. The method as claimed in claim 23, further comprising adding at least one active substance to the mixture during the first step.

30. The method as claimed in claim 29, wherein the active substance is selected from the group consisting of pharmaceutical active principles, active substances for cosmetic purposes, cells and DNA or RNA fragments.

31. The method as claimed in claim 23, wherein the volume fraction of polymer added to the mixture during the third step is between one and two times the total mass of the cationic and anionic surfactants.

32-33. (canceled)

34. The method of claim 30, wherein the membrane is a faceted hollow polyhedron.

35. The method of claim 30, wherein the cells are bacterial cells.

36. The method of claim 29, wherein the active substance is a chemical reactant, and further comprising diffusing reagents through pores of the catanionic membrane for chemical reactions with the chemical reactants.

37. The method of claim 36, wherein the catanionic membrane is in the form of a faceted hollow polyhedron

38. The method of claim 36, wherein the chemical reactions comprise precipitation and/or crystallization.

39. The method of claim 34, wherein the active substance is a cosmetic, and further comprising:
flocculating the membrane in the form of bunches of polyhedra; and
absorbing the polyhedra onto surfaces of opposite surface electrical potential, thereby allowing efficient diffusion of the medicament.

40. The method of claim 39, wherein the medicament is a cosmetic cream.

41. A method of retaining volatile molecules by absorption and slow diffusion comprising mixing said volatile molecule with the catanionic membrane of claim 1, whereby the volatile molecule is retained.

42. A method of vectorizing an active species comprising mixing the active species with the catanionic membrane of claim 1, whereby the active species is vectorized.

43. The method of claim 35, wherein said cells are encapsulated.

44. The method of claim 30, wherein the active substance is DNA or RNA fragments and wherein said fragments are encapsulated.

45. The method of claim 44, wherein the membrane is a faceted hollow polyhedron.
